(19) United States
(12) Patent Application Publication
(10) Pub. No.: US 2015/0190453 A1
(43) Pub. Date: Jul. 9, 2015

(54) Herbal Composition and a Method for the Treatment of Hyperlipidemia Caused by Excessive Consumption of Alcohol

(71) Applicant: Shuai Guo, Jinan (CN)

(72) Inventor: Shuai Guo, Jinan (CN)

(21) Appl. No.: 14/586,008

(22) Filed: Dec. 30, 2014

(30) Foreign Application Priority Data
Jan. 5, 2014 (CN) 201410002838.4

(51) Int. Cl. A61K 36/906; A61K 36/28; A61K 45/06; A61K 36/29; A61K 36/47; A61K 36/896; A61K 36/82; A61K 36/74

(52) U.S. Cl. CPC A61K 36/906; A61K 36/82; A61K 36/28; A61K 36/74; A61K 36/29; A61K 36/47; A61K 36/896; A61K 45/06

(57) ABSTRACT

A composition and a method for treating hyperlipidemia caused by excessive consumption of alcohol. The composition comprises in various parts: folium mahoniae, tender catchweed bedstraw herb, common dysosmatis rhizome and root, root of anhwei barberry, folium isatidis, garden erphorbia herb, dogtooth violet, climbing groundsel herb, huperzia serrata, puff-ball, meadowrue root and rhizome, herba portulacae, Chinese iris seed, European verbena herb, wild buckwheat rhizome, filliform cassytha herb, fig, shrubalthea bark, cottonrose hibiscus flower, cortex fraxini, Japanese white birch bark, peel of bottle gourd, corm of waternut, plumula nelumbinis, watershield, spica prunellae, herba commelinae, copperleaf herb, herba centellae, rhizoma bistortae, fructus gardeniae, radix scutellariae, rhizoma coptidis, cortex phellodendri, tea leaves, herb of spanishneedles, and radix glycyrrhizae. The method comprises weighing each aforementioned herb, blending them into water and soak, boiling the mixture, and filtering the residue.

The present application hereby claims priority to Chinese application No. 201410002838.4, filed on Jan. 5, 2014.

### TECHNICAL FIELD

The present invention relates to Chinese medicine compositions and a method for medical preparation, and more specifically speaking, it is a preparation for, and a method for the treatment of hyperlipidemia caused by excessive consumption of alcohol.

### BACKGROUND OF THE INVENTION

The name of “hyperlipidemia” does not exist in traditional Chinese medicine (“TCM”), but it has long been recognized and referred as “phlegm,” “fat,” “stroke,” “vertigo,” and other diseases under the “dirty blood disease” category. According to TCM, “dirty” blood is loaded with toxins or waste products, which is caused by turbid Qi, phlegm dampness and stasis in blood. It is recorded in Confucian's Duties To their Parents that, “people who eat fine food such as fat and refined grains, will be harmed by food and drinks they consumed, and may develop flatulence, fullness and heartburn.” TCM believes that one of the causes of this disease is patients' excessive consumption of alcohol, dysfunction of spleen, excessive accumulation of lipid, and the disruption of lipid's transportation, usage, and excretion within the human body.

Nowadays, lipid regulating drugs that are generally used for the treatment of hyperlipidemia caused by excessive consumption of alcohol are listed as follows:
1. Vitamin E nicotinate: Adverse reactions may include warm feelings on neck and facial area, skin redness, headaches and other reactions. Patients may also experience serious skin flushing, itching and gastrointestinal discomfort. Patients with arterial bleeding, diabetes, glaucoma, gout, hyperuricemia, liver diseases, ulcer diseases, and hypotension should use the product with caution.
2. Acipimox: Initial treatment can cause skin vasodilation, increase sensitivity to heat, such as hot flashes over the face or itching all over the body which would usually disappear in a few days after treatment, without stopping the medication. It shall not be used in patients who are allergic to this drug, or who have gastrointestinal ulcers. Pregnant women, lactating women, or children are forbidden to use this drug. Before using this medicine, patients should have low-cholesterol and low-fat diets and quit drinking Dosages shall be reduced in patients with renal insufficiency.
3. Bezafibrate: The most common adverse reactions were gastrointestinal discomfort, such as indigestion, anorexia, nausea, vomiting, and a sense of fullness. It shall not be used in patients who are allergic to the drug, or in patients who suffer from gallbladder diseases, cholelithiasis, hepatic insufficiency or primary biliary cirrhosis, severe renal insufficiency, and hypoalbuminemia caused by nephrotic syndrome. It shall be used with caution in pregnant and lactating women.
4. Fenofibrate: The probability of occurrence of adverse reactions is about 2% -15%, among which gastrointestinal reactions and insomnia are the common reactions. Contraindications include patients with known allergic reactions to this drug, a history of gallbladder disease, cholelithiasis, severe renal insufficiency, hepatic dysfunction, primary biliary cirrhosis, or unexplained chronic liver function abnormalities. It shall be used with caution in patients with gallstone, liver dysfunction and renal insufficiency. Patients need to stop using the product if serum amylase, or blood cholesterol, low-density lipoprotein level becomes elevated while using. This product is not a substitute for diet therapy, and diet control is still necessary. If the product has to be used in combination with hydroxy methyl coenzyme A (HMG-CoA) reductase inhibitors, the patients shall be closely monitored for serum creatine kinase (CK) levels. In case of significantly increased CK values or suspected myopathy or rhabdomyolysis, the usage of the product shall be immediately stopped. Patients shall be informed to consult a doctor immediately when they experience muscle pain, tenderness, or muscle weakness because sometimes they lead to very serious consequences.
5. Fluvastatin: Adverse reaction is a mild and transient indigestion. Patients who suffer active hepatitis or unexplained persistent elevation in serum transaminase, pregnant and lactating women, as well as patients under 18 years old are forbidden to use this product. Alcoholics, patients with a past history of liver diseases, and those who are allergic to other HMG-CoA reductase inhibitors are suggested to use the product with caution. When this product is used for treatment, diet therapy must be carried out simultaneously. In case of persistent elevation in serum transaminases, and more than three times over the upper limit, or significantly increased creatine kinase or myositis, this drug shall be discontinued immediately.
6. Gemfibrozil: The most common side effect is gastrointestinal discomfort, such as indigestion, anorexia, nausea, vomiting, fullness, and stomach discomfort. Patients with known allergic reactions to this drug, or patients who suffer from gallbladder disease, cholelithiasis, liver dysfunction or primary biliary cirrhosis, severe renal insufficiency, and reduction of serum proteins caused by nephrotic syndrome are forbidden to use the product. The product also interferes with the diagnosis, which may lead to a reduction of hemoglobin, hematocrit, or white blood cells, or an elevation of blood creatine phosphokinase, alkaline phosphatase, aminotransferase, or lactate dehydrogenase. After discontinuing the use of the product, the blood cholesterol and triglyceride levels may rebound over the original levels, so that a low-fat diet shall be prescribed and blood lipids shall be monitored until they reach normal levels.
7. Lovastatin: The most common side effect is gastrointestinal discomfort, such as, diarrhea and flatulence, and also includes headaches, rashes, dizziness, blurred vision and taste disorders. It can cause reversible elevated serum aminotransferase. Hence it is necessary to monitor patients' liver functions. Patients with known allergic reactions to this product, or patients who have active hepatitis or unexplained persistent elevation in serum aminotransferase are forbidden to use the product. Patients who are allergic to other HMG-CoA reductase inhibitors should use this product with caution. In case of an elevation of serum aminotransferase more than three times over the upper limit while using the product, or a significant increase of creatine kinase or myositis and pancreatitis, patients shall discontinue the usage immediately. When using this product, if patients experience hypotension, severe acute infection, trauma or metabolic disorders, it shall be noted that renal failure may occur secondary to myolysis.
8. Pravastatin: Adverse reactions include mildly elevated aminotransferase, rash, myalgia, headache, chest pain, nausea, vomiting, diarrhea, fatigue and so on. Patients with known allergic reactions to this drug, or patients who have active hepatitis or persistent elevation in liver function tests, as well as pregnant and lactating women are forbidden to use the drug. Patients with a history of liver disease or drinking history, as well as patients who are allergic to other HMG-CoA reductase inhibitors shall use the product with caution. It has poor efficacy for treating patients who have homozygous familial hypercholesterolemia. During treatment, liver function should be checked regularly. If persistent serum alanine aminotransferase test (ALT) or aspartate transaminase (AST) elevation equals or exceeds three times over the upper limit, treatment shall be discontinued. Use of HMG-CoA reductase inhibitor type of lipid-lowering drugs may occasionally cause elevated creatine phosphokinase (CPK). If the elevated value of CPK is ten times over three times of the normal upper limit, the usage of the product shall be discontinued. In case of unexplained muscle pain, tenderness, and weakness, especially accompanied by malaise and fever, attentions should be drawn. In case of hypotension, severe acute infection, trauma and metabolic disorders when using this product, it shall be noted that renal failure may occur secondary to myolysis.
9. Simvastatin is generally well tolerated with mild and transient adverse reactions in most patients. It is prohibited to be used by patients who are allergic to any component, pregnant women, or patients with active hepatitis or unexplained persistent elevation in serum aminotransferase. It shall be used with caution if used in combination with the tetrahydronaphthol type of calcium channel blocker Mibefradil. It shall be used with caution in patients who consume excessive alcohol or have a previous history of liver disease. The conditions of diffused muscle pain, muscle weakness and/or significant CK elevation (more than ten times greater than normal value) should be considered to be myopathy, and the treatment shall be immediately discontinued. For patients with acute or serious conditions suggestive of myopathy and a tendency of acute renal failure caused by rhabdomyolysis, HMG-CoA reductase inhibitor treatment shall be discontinued. Due to complete lack of low-density lipoprotein (LDL) receptors in patients with homozygous familial hypercholesterolemia, the effect of simvastatin treatment is not ideal for such patients.
10. Alginic Sodium Diester: The probability of occurrence of adverse reactions is 5%-23%, which mainly are palpitations, angina pectoris, hypotensions and electrocardiography (ECG) abnormalities. Possible adverse reactions also include xerostomia, nausea, vomiting, diarrhea, abdominal pain, constipation and loss of appetite. In some cases, patients may also develop liver abnormalities manifested as elevation in thymol turbidity test (TTT) or ALT, skin redness, itching, rash, annular erythema and erythroderma, extremity venous varix, limb peripheral edema, acute laryngeal edema and anaphylactic shock. It shall not be used in patients with bleeding disorder or bleeding tendency, or severe liver and kidney dysfunction. It shall be used with caution in patients with hypotension, hypovolemia, thrombocytopenia, non-hyperviscosity syndrome, non-platelet aggregation hyperactivity, and allergic constitution.

The adverse effects of the above treatments, and the usage limitations in certain group of patients are overcome by the present invention.

### SUMMARY OF THE INVENTION

To overcome the aforementioned shortcomings, the present invention is to provide a medical composition and a method for the treatment for hyperlipidemia caused by excessive consumption of alcohol. Here, hyperlipidemia refers to lipid metabolism disorders, abnormal fat metabolism or transport, too high lipid concentration in plasma, including total cholesterol and triglyceride levels, higher than those of normal people of the same age. The clinical conditions of hyperlipidemia caused by excessive consumption of alcohol are including but not limited to excessive consumption of alcohol together with excessive consumption of fat and sweets, overweight, dizziness, hypersomnia, and yellow-coated tongue.

Unlike Western medicines, TCM formulas comprise many components, usually extract of raw herbs with each one present in a very small quantity. The belief is that there is synergism among different components. One advantage of this kind of formulation is to avoid excessive toxicity when any one component is given in large quantity. The present invention is prepared by blending forty-six kinds of herbs that are common and can be purchased in any TCM pharmacy. The finished liquid medicine can be easily made and has minimal side effects, with a shorter course of treatment and a higher cure rate. These herbs assist and guide each other, and could avoid the adverse effects, allergic reactions and toxicity caused by antibiotics in Western medicine.

To obtain the forgoing object, the technical solution is provided as follows. An herbal composition, and a method for treatment of hyperlipidemia caused by excessive consumption of alcohol most preferably comprises by weight: 5 parts of folium mahoniae, 6 parts of tender catchweed bedstraw herb, 6 parts of common dysosmatis rhizome and root, 6 parts of root of anhwei barberry, 6 parts of folium isatidis, 5 parts of garden erphorbia herb, 6 parts of dogtooth violet, 6 parts of climbing groundsel herb, 6 parts of huperzia serrata, 2 parts of puff-ball, 6 parts of meadowrue root and rhizome, 6 parts of herba portulacae, 6 parts of Chinese iris seed, 6 parts of European verbena herb, 5 parts of wild buckwheat rhizome, 6 parts of filliform cassytha herb, 8 parts of fig, 6 parts of shrubalthea bark, 6 parts of cottonrose hibiscus flower, 6 parts of cortex fraxini, 5 parts of Japanese white birch bark, 6 parts of peel of bottle gourd, 2 parts of corm of waternut, 6 parts of plumula nelumbinis, 1 parts of watershield, 6 parts of spica prunellae, 2 parts of herba commelinae, 6 parts of copperleaf herb, 6 parts of herba centellae, 6 parts of rhizoma bistortae, 6 parts of fructus gardeniae, 5 parts of radix scutellariae, 6 parts of rhizoma coptidis, 6 parts of cortex phellodendri, 6 parts of tea leaves, 6 parts of herb of spanishneedles, and 9 parts of radix glycyrrhizae.

The processed method of the preparation of TCM for the treatment of hyperlipidemia caused by excessive consumption of alcohol comprises the steps as follows: (a) Providing the raw materials as follows: 5-10 parts of folium mahoniae, 6-12 parts of tender catchweed bedstraw herb, 6-12 parts of common dysosmatis rhizome and root, 6-12 parts of root of anhwei barberry, 6-12 parts of folium isatidis, 5-10 parts of garden erphorbia herb, 6-12 parts of dogtooth violet, 6-12 parts of climbing groundsel herb, 6-12 parts of huperzia serrata, 2-4 parts of puff-ball, 6-12 parts of meadowrue root and rhizome, 6-12 parts of herba portulacae, 6-12 parts of Chinese iris seed, 6-12 parts of European verbena herb, 5-10 parts of wild buckwheat rhizome, 6-12 parts of filliform cassytha herb, 8-16 parts of fig, 6-12 parts of shrubalthea bark, 6-12 parts of cottonrose hibiscus flower, 6-12 parts of cortex fraxini, 5-10 parts of Japanese white birch bark, 6-12 parts of peel of bottle gourd, 2-4 parts of corm of waternut, 6-12 parts of plumula nelumbinis, 1-2 parts of watershield, 6-12 parts of spica prunellae, 2-4 parts of herba commelinae, 6-12 parts of copperleaf herb, 6-12 parts of herba centellae, 6-12 parts of rhizoma bistortae, 6-12 parts of fructus gardeniae, 5-10 parts of radix scutellariae, 6-12 parts of rhizoma coptidis, 6-12 parts of cortex phellodendri, 6-12 parts of tea leaves, 6-12 parts of herb of spanishneedles, and 9-18 parts of radix glycyrrhizae; (b) put the provided raw materials into 1800 ml of water, and soak for 30 minutes; (c) boil the medicinal herbs with gentle heat for 30 minutes; (d) filter the residue to achieve 390 ml of liquid, which is the Chinese medicine for treatment of hyperlipidemia caused by excessive consumption of alcohol. When a patient needs treatment for hyperlipidemia caused by excessive consumption of alcohol, the drug liquid shall be adjusted to 35° C. He or she needs to consume 130 ml of liquid medicine each time, three times a day, and two days for one treatment course. If symptoms still exist, a second course of treatment is required until curing.

Among the thirty-seven kinds of herbs, folium mahoniae, tender catchweed bedstraw herb, common dysosmatis rhizome and root, root of anhwei barberry, folium isatidis, garden erphorbia herb, dogtooth violet, climbing groundsel herb, huperzia serrata, puff-ball, meadowrue root and rhizome, herba portulacae, Chinese iris seed, European verbena herb, wild buckwheat rhizome, filliform cassytha herb, fig, shrubalthea bark, cottonrose hibiscus flower, have the effect of inducing diuresis for removing edema, speeding up the metabolism and clearing toxic materials, relieving fidgets, and reducing pain. These nineteen herbs play a predominant role in treating hyperlipidemia caused by excessive consumption of alcohol, so they are principal drugs.

Cortex fraxini, Japanese white birch bark, peel of bottle gourd, corm of waternut, plumula nelumbinis, watershield, spica prunellae, herba commelinae, copperleaf herb, herba centellae, rhizoma bistortae, fructus gardeniae, radix scutellariae, rhizoma coptidis, cortex phellodendri, have the effects of replenishing vital essence, regulating the digestive system, invigorating spleen and normalizing the function of stomach, stopping sweating and inducing salivation, as well as pain relieving. These fifteen herbs could assist the principal drugs in treatment of hyperlipidemia caused by excessive consumption of alcohol, so they are ministerial drugs.

Traditionally, tealeaves and spanishneedles are believed to be able to lead the other herbs to diseased tissues, so they are used as an adjuvant drug. Radix glycyrrhizae could relieve spasm and pain, clear away toxic materials, and coordinate the nature of drugs. It is believed to be able to reduce the toxicity of various drugs, so it is used as a guiding drug.

These thirty-three herbs are applied with compatibility, together achieving the effects of removing edema, clearing away heat and toxic materials, reducing dampness jaundice, inducing diuresis for treating stranguria, clearing heat and relieving fidgets, strengthening the spleen and nourishing the stomach, clearing liver and improving vision, nourishing blood to tranquilize the mind, invigorating the spleen and normalizing the function of the stomach as well as relieving pain. With proper compatibility in these herbs, this drug is the best formula for the treatment of hyperlipidemia caused by excessive consumption of alcohol.

### EXAMPLES

There are 399 patients with hyperlipidemia caused by excessive consumption of alcohol in total, 226 males, accounting for 56.6%, and 173 females, accounting for 43.4%.

TABLE 1
Age range for 399 patients with hyperlipidemia
caused by excessive consumption of alcohol

| Age | 25~30 | 31~35 | 36~40 | 41~51 |
|---|---|---|---|---|
| Number of cases | 115 | 139 | 132 | 13 |
| Percentage | 28.8% | 34.8% | 33.1% | 3.3% |

TABLE 2
Efficacy in 399 patients with hyperlipidemia
caused by excessive consumption of alcohol

| Efficacy | Cured | Improved | Invalid |
|---|---|---|---|
| Number of cases | [symbol] 96 | 3 | No |
| Percentage | [symbol] 9.2% | 0.8% | No |

[symbol]

indicates data missing or illegible when filed

TABLE 3
Treatment course for 398 patients with hyperlipidemia
caused by excessive consumption of alcohol

| Treatment days | 3~5 | 6~9 | 10~14 | 15~23 |
|---|---|---|---|---|
| Number of cases | 152 | 109 | 127 | 11 |
| Percentage | 38.1% | 27.3% | 31.8% | 2.8% |

TABLE 4
Before and after treatment in 398 patients with hyperlipidemia caused by excessive consumption of alcohol

| | | | After treatment | | |
|---|---|---|---|---|---|
| | | Before treatment | Cured | Improved | Invalid |
| Clinical | | Excessive consumption of | No consumption of | Occasional consumption | Same with |
| conditions | | alcohol together with fat | alcohol, dizziness, | of alcohol, dizziness, | the conditions |
| | | and sweets, overweight, | hypersomnia, or yellow- | hypersomnia, and yellow- | before treatment |
| | | dizziness, hypersomnia, | coated tongue. | coated tongue. | |
| | | and yellow-coated tongue. | | | |
| Blood | Total | Increase | Normal | Slight increase | Same with |
| test | cholesterol | | | | the conditions |
| | | | | | before treatment |
| | Triglyceride | Increase | Normal | Slight increase | Same with |
| | | | | | the conditions |
| | | | | | before treatment |

What is claimed is:

1. A composition for treating hyperlipidemia caused by excessive consumption of alcohol, wherein the hyperlipidemia refers to lipid metabolism disorders, abnormal fat metabolism or transport, too high lipid concentration in plasma, including total cholesterol and triglyceride levels, higher than those of normal people of the same age, the said composition comprising:
i. folium mahoniae in an amount of 5-10 parts by weight,
ii. tender catchweed bedstraw herb in an amount of 6-12 parts by weight,
iii. common dysosmatis rhizome and root in an amount of 6-12 parts by weight,
iv. root of anhwei barberry in an amount of 6-12 parts by weight,
v. folium isatidis in an amount of 6-12 parts by weight,
vi. garden erphorbia herb in an amount of 5-10 parts by weight,
vii. dogtooth violet in an amount of 6-12 parts by weight,
viii. climbing groundsel herb in an amount of 6-12 parts by weight,
ix. huperzia serrate in an amount of 6-12 parts by weight,
x. puff-ball in an amount of 2-4 parts by weight,
xi. meadowrue root and rhizome in an amount of 6-12 parts by weight,
xii. herba portulacae in an amount of 6-12 parts by weight,
xiii. Chinese iris seed in an amount of 6-12 parts by weight,
xiv. European verbena herb in an amount of 6-12 parts by weight,
xv. wild buckwheat rhizome in an amount of 5-10 parts by weight,
xvi. filliform cassytha herb in an amount of 6-12 parts by weight,
xvii. fig in an amount of 8-16 parts by weight,
xviii. shrubalthea bark in an amount of 6-12 parts by weight,
xix. cottonrose hibiscus flower in an amount of 6-12 parts by weight,
xx. cortex fraxini in an amount of 6-12 parts by weight,
xxi. Japanese white birch bark in an amount of 5-10 parts by weight,
xxii. peel of bottle gourd in an amount of 6-12 parts by weight,
xxiii. corm of waternut in an amount of 2-4 parts by weight,
xxiv. plumula nelumbinis in an amount of 6-12 parts by weight,
xxv. watershield in an amount of 1-2 parts by weight,
xxvi. spica prunellae in an amount of 6-12 parts by weight,
xxvii. herba commelinae in an amount of 2-4 parts by weight,
xxviii. copperleaf herb in an amount of 6-12 parts by weight,
xxix. herba centellae in an amount of 6-12 parts by weight,
xxx. rhizoma bistortae in an amount of 6-12 parts by weight,
xxxi. fructus gardenia in an amount of 6-12 parts by weight,
xxxii. radix scutellariae in an amount of 5-10 parts by weight,
xxxiii. rhizoma coptidis in an amount of 6-12 parts by weight,
xxxiv. cortex phellodendri in an amount of 6-12 parts by weight,
xxxv. tealeaves in an amount of 6-12 parts by weight,
xxxvi. herb of spanishneedles in an amount of 6-12 parts by weight, and
xxxvii. radix glycyrrhizae in an amount of 9-18 parts by weight;

2. The composition of claim 1, wherein the composition is in a form of a concentrated oral solution.

3. A method for treating hyperlipidemia caused by excessive consumption of alcohol, the method comprising administrating an effective amount of a medicinal composition to a subject in need thereof, wherein said medicinal composition is prepared by a method comprising,
i. weighing predetermined amount of each of folium mahoniae, tender catchweed bedstraw herb, common dysosmatis rhizome and root, root of anhwei barberry, folium isatidis, garden erphorbia herb, dogtooth violet, climbing groundsel herb huperzia serrata, puff-ball, meadowrue root and rhizome, herba portulacae, Chinese iris seed, European verbena herb, wild buckwheat rhizome, filliform cassytha herb, fig, shrubalthea bark, cottonrose hibiscus flower, cortex fraxini, Japanese white birch bark, peel of bottle gourd, corm of waternut, plumula nelumbinis, watershield, spica prunellae, herba commelinae, copperleaf herb, herba centellae, rhizoma bistortae, fructus gardeniae, radix scutellariae, rhizoma coptidis, cortex phellodendri, tea leaves, 6-herb of spanishneedles, and radix glycyrrhizae.
ii. blending the provided raw materials into 1800 ml of water, and soak for 30 minutes;
iii. boiling the medicinal herbs with gentle heat for 30 minutes;
iv. filtering the residue to achieve 390 ml of liquid.
